(19) United States
(12) Patent Application Publication
(10) Pub. No.: US 2023/0128619 A1
(43) Pub. Date: Apr. 27, 2023

(54) TOLERANCE TO VIRUSES

(71) Applicant: ORIGENE SEEDS LTD, Givat Brenner (IL)

(72) Inventors: Eyal A. VARDI, Givat Brenner (IL); Moti SABAN, Givat Brenner (IL)

(21) Appl. No.: 17/913,493

(22) PCT Filed: Apr. 2, 2021
(86) PCT No.: PCT/IL2021/050370

§ 371 Date: Sep. 22, 2022

Related U.S. Application Data
(60) Provisional application No. 63/003,958, filed on Apr. 2, 2020.

(51) Int. Cl. C12N 15/82

(52) U.S. Cl. CPC C12N 15/8283

(57) ABSTRACT

A watermelon plant and seed, namely of Citrullus lanatus, which are resistant to geminiviruses, especially to begomoviruses including Watermelon chlorotic stunt virus (Wm CSV), comprising in their genome introgressed sequences from Citrullus colocynthis conferring tolerance to said viruses, when present homozygously. More specifically, the invention also discloses molecular genetic markers, linked to the recessive genetic locus conferring tolerance to geminiviruses comprising A Citrullus lanatus seed having in its genome introgressed sequences from Citrullus colocynthis conferring tolerance to geminiviruses only when present homozygously, wherein said introgressed sequences are located on homologous chromosome 6, within the chromosomal region delimited by nucleotides 1-2,255,000.

### TECHNOLOGICAL FIELD

The invention relates to watermelon plants. The present invention is directed to watermelon plant and seed, namely of Citrullus lanatus, which are resistant to geminiviruses, especially to begomoviruses including Watermelon chlorotic stunt virus (WmCSV), comprising in their genome introgressed sequences from Citrullus colocynthis conferring tolerance to said viruses, when present homozygously. More specifically, the invention also discloses molecular genetic markers, linked to the recessive genetic locus conferring tolerance to geminiviruses

### BACKGROUND

Watermelon crops (Citrullus lanatus), of the Cucurbitaceae family, are naturally-occurring diploids. The set of chromosomes of watermelon plants can undergo duplication and exist as tetraploids (i.e., having four sets of chromosomes). A tetraploid female parent can be crossed with a diploid male parent to produce triploid hybrid, having three sets of chromosomes, which cannot be divided equally during meiosis. A triploid hybrid plant produces a seedless watermelon fruit. A tetraploid plant produces a small amount of seeds compared to a diploid plant. Further, a substantial amount of seed yield is required for a commercial triploid hybrid product.

WmCSV is a virus that infects plants such as watermelon, melon (Cucumis melo L.) [Kheyr-Pour et al., 2000; Alhudaib et al., 2018] and cucumbers (Cucumis sativus). WmCSV is a bipartite member of the genus Begomovirus, belonging to the Geminiviridae family.

Begomoviruses are transmitted by whitefly (Bemisia tabaci), in a persistent manner Whitefly transmitted viruses have been held responsible for significant crop losses in many regions with tropical, sub-tropical, arid and Mediterranean climates. WmCSV disease incidence is associated with high population of this vector [Abudy et al., 2010; Al Musa et al., 2011].

Begomoviruses have spread rapidly amongst Cucurbits throughout the world. The disease has progressed to alarming levels in many eastern Mediterranean countries. Given the wide distribution of WmCSV in the eastern Mediterranean countries and the endemic population of the whitefly vector in the region, particular attention to the virus is required. Otherwise, invasion and remarkable yield losses to watermelons and to other Cucurbits crops sensitive to the Begomovirus is inevitable [Al Musa et al., 2011].

In Israel for example, the cost for controlling the virus is in average approximately 195 NIS per one dunam of watermelons (500 S per hectare) (Origene Seeds Internal data). According to this calculation, to control 165,000 hectares in Turkey, 82,500,000 S is required.

The appearance of the disease is recognized in leaves of infected plants which appear crinkled with yellow spots chlorotic mottling. Fruits are reduced in size with chlorotic blotches which may not develop to maturity. The cultivars grown in the affected regions show severe diseases symptoms [Al Musa et al., 2011].

In recent years watermelon has also been characterized at the DNA level and DNA markers from different linkage regions of the watermelon genome have been identified using various techniques, mainly by the use of PCR reaction techniques. Molecular markers are useful in identifying and isolating genes controlling disease resistance [Levi et al., 2006]. But, all of the markers that were obtained related to the isolated virus itself.

Three isolates of the virus have been obtained from Sudan, Iran and Yemen [Kehyr-Pour et al., 2000]. All three comprise two single stranded DNA molecules (DNA-A and DNA-B), processing a high degree of similarity. Whereas genes from DNA-A encode proteins involved in replication and regulation, DNA-B encodes proteins required for viral infection. Nucleotide sequence comparisons revealed that WmCSV isolate that originated from Israel, within watermelon plots near Eilat, is highly homologous to other WmCSV isolates, with the highest homology (nearly identical) to the isolate from Sudan [Abudy et al., 2010].

Resistance to the virus was found in wild watermelon, “C.c.” (Citrullus colocynthis) [Abudy et al., 2010]. This perennial watermelon source for the resistance is found in geographic locations of the wild watermelon, such as in Israel, North Africa deserts, the Sahara desert, South West Asia, India and worldwide geographical regions with similar climate.

### SUMMARY OF DISCLOSURE

In accordance with a first of its aspects, the present disclosure provides a hybrid or open pollinated or parental line watermelon plant characterized by:
tolerance to Watermelon Chlorotic Stunt Virus (WmCSV);
being a diploid, tetraploid or triploid plant;
seedless or seeded fruit having a total soluble solid of at least 9%, at times between 10 to 14%; and
Yellow, orange of red fruit flesh color, preferably dark red.

The present disclosure further provides seeds of a triploid watermelon plant that produces a watermelon plant described herein, namely a triploid watermelon hybrid plant characterized by tolerance to WmCSV, capable of producing seedless fruit with a total soluble solid of at least 9%, at times, between 10%-14%, and a red or red-like fruit flesh color.

The present disclosure also provides a seedless watermelon fruit obtained from the triploid hybrid watermelon plant disclosed herein or from its seeds.

In another of its aspects, the invention provides a tetraploid watermelon female plant characterized by having tolerance to WmCSV and within a score tolerance scale from 1 to 9, and by having seed yield of at least 4, at times at least 5, at times at least 6, and further at times, at least 7, at times at least 8. Preferably, a tolerance scale score of 7-9 is achieved as indicative of WmCSV tolerance.

In accordance with this aspect, there is also provided a tetraploid watermelon plant as well as any other offspring of the tetraploid watermelon. Further in accordance with this aspect, there is provided pollen of a tetraploid watermelon plant disclosed herein.

The present disclosure further provides a watermelon fruit obtained from the tetraploid watermelon plant line disclosed herein or from its seeds.

In accordance with this aspect, there is also provided seeds of diploid watermelon plant as well as any other offspring of the diploid watermelon. Further in accordance with this aspect, there is provided a pollen of a diploid watermelon plant disclosed herein.

In yet a further aspect, there is provided by the present disclosure, a method of producing a triploid hybrid watermelon plant characterized by:
tolerance to Watermelon Chlorotic Stunt Virus (WmCSV);
being a triploid plant;
a seedless fruit having a total soluble solid of at least 9%, at times between 10 to 14%; and
red fruit flesh color, preferably dark red,

the method comprising:
a. planting a tetraploid watermelon line being at least WmCSV tolerant and having high seed yield to obtain a tetraploid watermelon plant;
b. crossing said tetraploid line with a WmCSV tolerant diploid watermelon line to produce triploid seeds; and
c. planting said triploid seeds to produce triploid hybrid watermelon plant disclosed herein.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1B are images of a WmCSV sensitive watermelon plant with symptoms of yellow chlorotic spots (yellow dots) appearing on the leaves and stems (FIG. 1A, left plant) as compared to a healthy green WmCSV watermelon hybrid plant according to the present disclosure exposed to WmCSV virus but not showed symptoms (FIG. 1A, right plant); and a close-up of a WmCSV sensitive watermelon plant that showed symptoms of yellow chlorotic spots appearing on the leaves (FIG. 1B).

FIG. 2 is an image of wild type watermelon source, Citrullus colocynthis (C.c.), that is tolerant to WmCSV, having light green rind and dark green “broken” stripes (FIG. 2(I)), white flesh and brown seeds (FIG. 2(II)).

FIG. 3 is an image of a triploid hybrid watermelon fruit according to the present disclosure which is referred to herein by the designation 12.607-S14 having representative seeds deposited under Accession No. NCIMB 42400.

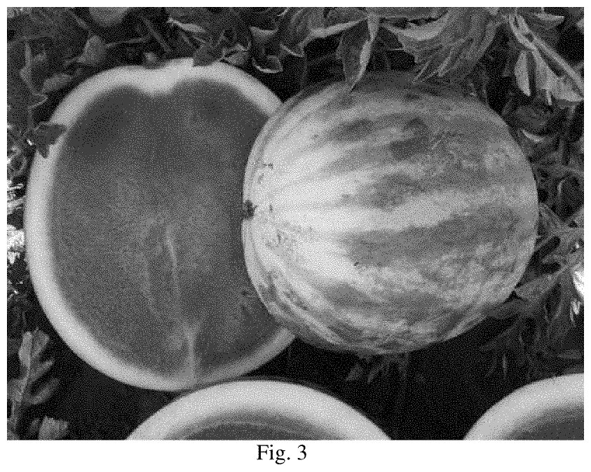

### EMBODIMENTS OF THE INVENTION

The following is a list of embodiments of the invention:
1. A watermelon plant characterized by:
   tolerance to Watermelon Chlorotic Stunt Virus (WmCSV);
   being a triploid plant;
   seedless fruit having a total soluble solid (TSS) of at least 9%; and
   red fruit flesh color.
2. The watermelon of Embodiment 1, having tolerance to WmCSV of between 5 to 9, in a tolerance scale of between 1 to 9.
3. The watermelon plant of any one of Embodiments 1 to 2, having a number of fruits per plant is between 2 and 4.
4. The watermelon plant of any one of Embodiments 1 to 3, characterized by a vigor between 60 and 90.
5. The watermelon plant of any one of Embodiments 1 to 4, characterized by fruit setting of between 50 and 90.
6. The watermelon plant of any one of Embodiments 1 to 5, characterized by an earliness of between 40 and 70.
7. Seeds of a triploid hybrid watermelon plant of any one of Embodiments 1 to 6.
8. The seeds of Embodiment 10, designated herein 12.607-S14 having representative seeds deposited under Accession No. NCIMB 42400.
9. A seedless watermelon fruit obtained from the triploid hybrid watermelon plant of any one of Embodiments 1 to 7 or from the seeds of Embodiment 7 or 8.
10. The watermelon fruit of Embodiment 9, characterized by one or more of the following parameters:
   stripped rind;
   rind width between 4 mm to 15 mm
   a round or oval shape;
   Orange, yellow or red flesh, preferably, dark red flesh;
   mini, midi or large size.
11. The watermelon fruit of Embodiment 10, characterized by an essentially round or oval shape and a red color flesh.
12. An offspring being asexually reproduced from the watermelon plant of any one of Embodiments 1-6 or seeds of Embodiment 7 or 8.
13. The watermelon plant of any one of Embodiments 1 to 6, obtained by crossing a WmCSV tolerant diploid watermelon plant, seeds or offspring thereof; with a WmCSV tolerant tetraploid watermelon plant, seeds or offspring thereof.
14. A tetraploid watermelon plant characterized by having tolerance to WmCSV and seed yield of at least 4 within a score scale from 1 to 9.
15. The tetraploid watermelon plant of Embodiment 14, having tolerance to WmCSV of at least 6 in a tolerance scale between 1 to 9.
16. Seeds of tetraploid watermelon plant of any one of Embodiments 14 to 15.
17. An offspring of the tetraploid watermelon plant of any one of Embodiments 14 to 15 or seeds of Embodiment 16.
18. Pollen of a tetraploid watermelon plant of any one of Embodiments 14 to 15.
19. A watermelon fruit obtained from the tetraploid watermelon plant of any one of Embodiments 14 to 15, from a seed of Embodiment 16 or from an offspring of Embodiment 17.
20. A method of producing a triploid watermelon hybrid plant of any one of Embodiments 1 to 6, the method comprising:
   a. planting a tetraploid watermelon line being at least WmCSV tolerant and having high seed yield to obtain a tetraploid watermelon plant;
   b. crossing said tetraploid line with a WmCSV tolerant diploid watermelon line to produce triploid seeds; and
   c. planting said triploid seeds to produce triploid hybrid watermelon plant having WmCSV tolerance and producing a seedless fruit having a total soluble solid of at least 9%.
      21. A method of producing the tetraploid watermelon plant of any one of Embodiments 14 to 15, the method comprising at least one backcross breeding between (i) a WmCSV sensitive tetraploid female line having high seed yield and (ii) a WmCSV tolerant tetraploid female line.
      22. A method of producing the tetraploid watermelon plant of any one of Embodiments 14 to 15 arising from a genome doubling, the method comprising at least one backcross breeding between (i) a WmCSV sensitive diploid female line having high seed yield and (ii) a WmCSV tolerant diploid female line.
      23. The tetraploid watermelon plant of any one of Embodiments 14 to 15, obtained by backcrossing of: (i) a high seed yield tetraploid watermelon plant sensitive to WmCSV seeds or offspring thereof and (ii) a low seed yield tetraploid watermelon plant having a tolerance to WmCSV of at least 6 in a tolerance scale of 1 to 9, seeds or offspring thereof.

### DETAILED DESCRIPTION OF EMBODIMENTS

The present disclosure is based, inter alia, on the understanding that in order to obtain a triploid watermelon plant with tolerance to WmCSV, both parent lines need to be resistant or at least with significant tolerance to the virus in order to transfer the trait to the progeny, to an extent that the progeny has sufficient tolerance, along with other desired characteristics that render the progeny edible.

The term “Resistance” as used herein, shall include but will not be limited to a resistance as defined by the ISF (International Seed Federation) Vegetable and Ornamental Crops Section for describing the reaction of plants to pests or pathogens, and abiotic stresses for the Vegetable Seed Industry and the ability of a plant variety to restrict the growth and development of a specified pest or pathogen and/or the damage they cause when compared to susceptible plant varieties under similar environmental conditions and pest or pathogen pressure, wherein resistant varieties may exhibit some disease symptoms or damage under heavy pest or pathogen pressure.

The term “Tolerance” is used herein, shall include but will not be limited to a an indication of a phenotype of a plant wherein disease-symptoms remain absent upon exposure of said plant to an infective dosage of virus, whereby the presence of a systemic or local infection, virus multiplication, at least the presence of viral genomic sequences in cells of said plant and/or genomic integration thereof can be established. Tolerant plants are therefore resistant for symptom expression but symptomless carriers of the virus. Sometimes, viral sequences may be present or even multiply in plants without causing disease symptoms.

In addition, it has been found that in order to obtain high seed production of said triploid watermelon plants with sufficient sweetness (to render it edible), appealing flesh color (red or red like) and the desired tolerance, a further backcrossing stage of the female parent is required. This backcrossing provided a significant and unexpected increase in the seed production of the hybrid seeds (produced by the tetraploid female).

Based on the extensive research conducted by the inventors, seedless hybrid triploid watermelon seeds and plant lines tolerant to WmCSV were developed. The triploid plants were produced by crossing a tetraploid line with high seed yield as the female parent with a diploid line as the male parent; the female parent being manipulated to be WmCSV tolerant while the tolerance of the diploid originated from the wild-type resistant plant as further described below.
Thus, in accordance with a first of its aspects, the present disclosure provides a watermelon plant hybrids characterized by at least:
   tolerance to Watermelon Chlorotic Stunt Virus (WmCSV);
   being a triploid hybrid plant;
   a seedless fruit having a total soluble solid of at least 9%, at times in the range of 10% to 14%; and
   red fruit flesh color.

In the context of the present disclosure the following terms are to be understood as having the following meanings:

Watermelon Chlorotic Stunt Virus (WmCSV)—WmCSV is to be understood as having the meaning known in the art, namely, a virus of the genus Begomovirus that is capable of infecting plant species such as, watermelon (Citrullus lanatus), characterized by chlorotic mottling on leaves of infected plants. As appreciated, fruits are reduced in size with chlorotic blotches which do not develop to maturity. Further symptoms can include any one of yellow veins, severe stunting of young leaves and drastically reduced fruit yield. In some embodiments, the WmCSV is an infection caused or transmitted by a whitefly Bemisia tabaci (Gennadius).

In some embodiments, the WmCSV infection of a plant is identified by any one or a combination of appearance on the plant of yellow chlorosis, appearing on the apex of the plant. In some embodiments, the yellow chlorotic spots appear on the young leaves underneath the apex. In some embodiments, the WmCSV infection is identified by the appearance of chlorotic spots being spread on various areas of the plant, including the stem and/or the fruit. In some embodiments, infection is identified by the appearance of necrotic areas. As appreciated, once chlorosis symptoms start typically after 10-15 days from the infection, it may cause death of the plant in a couple (several) of weeks.

WmCSV tolerance—in the context of the present disclosure, tolerance (or lack of sensitivity) is ranked between 1 to 9, with 1 defining a high degree of sensitivity (no tolerance) that can even result in severe chlorosis symptoms, and 9 defining high tolerance or even resistance and no symptoms at all. Thus, it is be understood that a sensitive plant has low tolerance to WmCSV and lack of sensitivity is indicative of tolerance or resistance to WmCSV. Accordingly, in the context of the present disclosure a plant ranked between 6-7 is regarded as tolerant, a plant ranked 7-8 is regarded with high tolerance or intermediate resistance, and a plant ranked 8-9 is regarded as resistant. One can be find a scale reference (0-3) in Adudy al al., 2010 as follows:

### 0 in Abudy et al., 2010 is 9, 1 is 6-8, 2 is 4-6 and 3 1-3.

In some embodiments, when referring to tolerance, intermediate resistance or tolerance, it is to be understood as referring to a watermelon plant comprising a tolerance locus being linked to a genetic background or genetic determinant obtainable from the genome of a wild type watermelon plant, Citrullus colocynthis (referred herein “C.c.”) being agriculturally recognized as having tolerance to WmCSV (scale of tolerance above 7, and preferably above 8), such as the C.c. watermelon plant designated according to the plant index 1-9 (Origene Seeds internal index: 1 is most sensitive and 9 is 100% resistant).

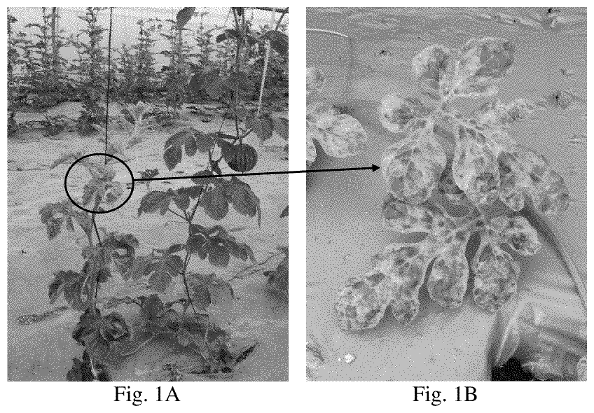

A comparison between WmCSV sensitive line (ranked as 2) and WmCSV tolerant line (ranked as 7-8) can be seen in FIGS. 1A and 1B, where the left side of FIG. 1A displays an image of a WmCSV sensitive watermelon plant with symptoms of yellow chlorotic spots appearing on the leaves (circled) and stems as compared to a healthy green WmCSV watermelon triploid hybrid plant according to the present disclosure, after being exposed to WmCSV virus but not showed symptoms (FIG. 1A, right plant). FIG. 1B is a close-up of leaves of the WmCSV sensitive watermelon plant with symptoms of yellow chlorotic spots appearing on the leaves of a plant.

Also and parallel, sample leaves taken from infected plants were sampled and tested for the presence of the viral DNA using PCR method with primers designed to detect DNA of begomoviruses. Leaves samples taken from plants with high tolerance score (i.e. the plants were inoculated with the virus but did not show any symptoms) did not show any PCR amplification for the presence of begomovirus viral DNA according to Rojas, 1993.

Seedless—In the context of the present disclosure it is to be understood that a seedless plant is defined by lack or insufficient number (not more than 20 seeds) of mature seeds in its fruit and thus lacking the capacity to propagate via seeds. It is to be understood that a seedless fruit can include seed coats and with respect to the present disclosure a seedless fruit is one comprising between 0-200 seed coats.

Fruit shape—Generally, a watermelon fruit can have the following shape; a flat, round, oval and elongated shape. The shape can be defined by the ratio between the major and minor axes of the fruit, such that an oval shape is defined by a ratio between the major axis 1.2-1.4 to the minor axis 1 as a ratio between 1.2:1 to 1.4:1 while in an elongated fruit the ratio is define between 1.5:1 and 1.8:1. Equally the shape may be in the range of 10 to 90, with 10 representing a flat fruit, 50 representing a round fruit, 60-70 represents an oval fruit and 75-90 represents an elongated fruit. This scale is calculated based on the fruit's measurements ratio (length/width)×50 (a round fruit).

Fruit regularity—in the context of the present disclosure is ranked between 10 to 90, wherein 10 indicates an irregular shape and 90 indicates that the fruit is regular in shape.

Vigor—in the context of the present disclosure watermelon plants can be characterized by their overall condition or their “vigor”, the vigor being scaled between 10 to 90; 10 defining a weak vigor and 90 defining a very strong vigor. Vigor is determined based on plant size and leaf blade size.

Flowering period—in the context of the present disclosure flowering period, also understood as earliness of a plant is defined by the time from planting to the first female flower and time from flowering to fruit maturation. In accordance with the present disclosure, earliness, i.e. the female flowering time is scaled between 10 to 90; 10 defining a very early female flowering time and 90 defining a very late female flowering time. In the summer, time to early (10) female flowering is defined between 20-25 days after planting and late (90) is defined 40-45 days after planting.

Fruit setting—in the context of the present disclosure is defined as the occurrence of fruits setting in a plant, the setting being scaled between 10 to 90; 10 reflecting very few fruits per plant (i.e., up to 0.5 fruit per plant) and 90 reflecting a large number of fruits per plant (above 4).

Number of fruit per plant—The number of fruit per plant is a well appreciated parameter in determining quality of a plant. Good (commercially suitable) quality plant is defined as one having between 1 to 4 fruits per plant, preferably between 2 to 3 fruits per plant.

Flower sex—Flower can be monoecious (M), namely, a watermelon plant species in which male and female organs are found on the same plant but in different flowers, hermaphrodite (H), namely, a plant where the male and female organs are present on the same flower; and andromonoecious (AM), namely, a plant that has both hermaphrodite and male flowers.

Rind color—The color of the rind of the fruit can be from smooth gray, to striped fruit like brake green stripe (citron type), thin stripes (Jubilee type), dark medium stripes (crimson type) and dark wide stripes on light green background (All sweet type) to smooth light or dark green rind.

Striping—Watermelons can be distinguished by the appearance of stripes on the outer surface of the rind. The stripes may cover small portions of the fruit's rind, medium coverage and also may cover the majority of the fruit's rind. The stripes may be very thin, medium or very thick. Thus, the overall stripes coverage of the rind, may be defined by percentage, with low coverage being regarded as less or equal to 10% rind cover, medium coverage—50% cover, and high/thick coverage—70-80% rind cover and very high/thick coverage—90% rind cover. The stripes are typically green stripes.

Weight—Watermelon fruits are typically characterized by their weight. Small fruits are those typically weighting less than 4 kg, at times, between 1.5 and 4 kg, and at times, between 2-4 kg, and are recognized as the mini or personal sized watermelons, and large watermelons are those weighting at least 4 kg. The group of large fruits may be further divided into two sub groups of sizes 4 kg-7 kg (also referred to as midi sized fruits) and at least 7 kg (also referred to as big sized fruits). Thus, in the context of the present disclosure, the fruits may be mini, having a weight of less than 4 kg, between 2 and 4 kg; midi, having a weight of between 4 and 6 kg; and large fruits having a weight larger than 7 kg, such as between 7 kg to 14 kg or 7 kg to 10 kg.

Flesh color—Generally, watermelon flesh color may vary from scarlet red (dark red), coral red (light red), orange, salmon yellow, canary yellow, or white. In the context of the present disclosure, when referring to red, it is to be understood to cover also shades or hues of red, including dark red or red-like color. The flesh color may be determined using Pantone Color scale and in accordance with some embodiments, the red color is identified by any one of the Pantone color scale available on line at www.pantone.com (2012) as Pantone 18-1664 TCX Fiery Red (i.e., dark red) to Pantone Red 032C (i.e., light red). The intensity of the redness of the flesh of the watermelon fruit described herein can be also referred as R5 and R6 color, in a scale of R1 to R6, where R1 being very light red and R6 being very deep red or very dark red.

Flesh firmness—Flesh firmness defines the force necessary to break the flesh tissue at ripening. Firmness of the watermelon plants disclosed herein was measured by Penetrometer Fruit Pressure Tester Mod. FT 011 (0-11 lbs.), IRC, which measures the pressure necessary to force a plunger of specified size into the pulp of the fruit. According to methodology known in the art, the measurement are obtained using specialized tips for each type of fruit being tested—3 mm (berries, grapes and other small fruits), 6 mm (avocados, berries), 8 mm (pears, stone fruit, avocados) and 11 mm (apples). Such pressure is measured in pounds or kilograms. The scale in accordance with the present disclosure was ranked between 10 to 90 (50 being 2 lbs), where 10 indicates that the flesh is very soft (e.g. penetrometer scale below 2), and 90 indicates a very firm flesh (e.g. penetrometer scale above 4). Penetrometer scale between 3-3.5 is considered firm flesh.

Optionally, when using an 8 mm tip, flesh firmness to a weight/pressure was ranked between 3 ounces to 31 ounces, where 3 ounces indicates that the flesh is very soft and 31 ounces indicates a very firm flesh. In some embodiments, the triploid hybrid watermelon disclosed herein, is characterized by fruit flesh firmness to pressure of 20-27 ounces when using an 8 mm tip, at times 24 ounces.

Rind thickness—Is usually measured in millimeters from the outer edge of the fruit to the boundary between the white mesocarp and colored endocarp. The thickness may vary from very thick, e.g. >19 mm, medium thickness 10-19 mm, to low thickness <10 mm.

Rind crack—Rind crack defines the tendency of the watermelon rind to crack by itself and in accordance with the present disclosure, rind cracks are ranked from 10 to 90, where 10 defines a strong, uneasily cracked rind (no tendency to crack at all) and 90 defines an easily cracked rind (the fruit is cracking by itself in the field).

Taste—While being somewhat a subjective characteristic, a fruit of a watermelon may vary from tasteful, tasteless, bitter etc.

TSS (Total Soluble Solids)—Is a measure of the percent soluble solids (TSS) in a sample of a plant juice, used as an index/parameter for sugar quantity in the fruit, namely, for determining the sweetness of the fruit. The TSS was measured by ATAGO refractometer (http://www.atago.net/USA/products_hsr.php), Brix 0.0 to 33.0%, Automatic Temperature Compensation, The refractometer designed to measure the refractive index of the solution. The Brix percentage represents the total concentration of total soluble solids (TSS) in the sample.

Seed production ability—In the context of the present disclosure it is to be understood as the overall fruit seed production ability of the female lines, being scaled between 1 to 9, where 1 defines low seed production ability and 9 defines very high seed production. The seed production ability of the female line was determined according to the number of seeds inside the female fruit according to the following scale: 1: less than 30 seeds, 2: between 30 to 50 seeds, 3: between 50-70 seeds, 4: between 70-90 seeds, 5: between 90 to 120 seeds, 6: between 12-150 seeds, 7: between 150-200 seeds, 8: between 200-240 seeds, 9: more than 240 seeds.

Deposit—Deposits of representative seeds deposited on 27 Apr. 2015 at the NCIMB recognized institute for purposes of patent procedure of the lines of Applicant's internal reference, 12.607-S14, and the following Accession number were provided: NCIMB 42400.

Deposits of representative seeds NCIMB 42400 included seeds developed as a new triploid hybrid line tolerant to WmCSV between 2012 and 2015 as detailed in table 1 hereinbelow for lines with Applicant's internal reference 12.607-S12, 12.607-W13, 12.607-S14 and 12.607-W14.

Hybrid—in the context of the present disclosure a hybrid plant is a plant produced by cross-pollination of plants in order to improve the characteristics of the plant, such as, but not limited to, better yield, greater uniformity, improved color, improved taste and disease tolerance.

Plant Breeding—plant breeding to modify the traits of a plant line (by crossing) in order to produce desired characteristics. Plant breeding may be accomplished by techniques depending on the mode of reproduction of a plant, such as, selection of plants with desirable characteristics for propagation, self-fertilization, cross-pollination, cultigen and cultivar.

Backcross Breeding—backcross breeding refers to a progeny of a cross that is further crossed with a parent having a specific trait to increase the possibility that the progeny will inherit a specific trait from that parent. When plants are crossed with themselves, the breeding produces inbred lines.

Turning to the embodiments of the present disclosure, according to a first of its aspects, the disclosure provides a watermelon plant characterized by:
tolerance to Watermelon Chlorotic Stunt Virus (WmCSV);
being a triploid plant;
seedless fruit having a total soluble solid of at least 9%, at times between 10 to 14%; and
Orange, yellow or red fruit flesh color, preferably dark red.

The triploid hybrid watermelon plant is characterized independently by any one of the properties described hereinbelow. Each one of the below properties may be taken alone or in combination with any one of the below properties.

In some embodiments, the triploid hybrid watermelon plant disclosed herein is characterized by tolerance to WmCSV of at least 5, at times, at least 6, at times, at least 7, at times, between 5 to 9, at times, between 6 to 9, at times, between 7 to 9, in a tolerance scale between 1 to 9.

In some embodiments, the triploid hybrid watermelon plant disclosed herein, is characterized by a number of fruits per plant is between 2 and 4.

In some embodiments, the triploid hybrid watermelon plant disclosed herein, is characterized by a vigor between 60 and 90.

In some embodiments, the triploid hybrid watermelon plant disclosed herein, is characterized by fruit setting of between 50 and 90.

In some embodiments, the triploid hybrid watermelon disclosed herein, is characterized by fruit rind color of crimson or crimson dark. In some other embodiments, the fruit rind color is characterized by dark green or gray stripes or tiger-type stripes. The triploid watermelon fruit can be further characterized by the stripes coverage of the fruit rind. In some embodiments, the stripes cover between 60-90% of the fruit rind, at times, at least 70%, at times at least 80%.

In some embodiments, the triploid hybrid watermelon disclosed herein, is characterized by red fruit flesh color, at times, deep or dark red fruit flesh color. These colors can be identified according to the Pantone Scale color as Pantone 18-1664 TCX Fiery Red (i.e., dark red) to Pantone Red 032C (i.e., light red). The intensity of the redness of the flesh of the watermelon fruit described herein can be also referred as being red to very deep red.

In some embodiments, the triploid hybrid watermelon fruit is characterized by fruit shape in the range of about 50 to 70 (namely, round or slightly oval), at times in the range of about 50 to 60, at times in the range of about 50-55. In some other embodiments, the triploid hybrid watermelon fruit is characterized by being essentially round. In some other embodiments, the fruit is oval.

In some embodiments, the triploid hybrid watermelon disclosed herein, is characterized by fruit flesh firmness between about 60-80, at times about 70.

In some embodiments, the triploid hybrid watermelon disclosed herein, is characterized by fruit flesh firmness to pressure of 20-27 ounces when using an 8 mm tip, at times 24 ounces.

In some embodiments, the triploid hybrid watermelon plant disclosed herein, is characterized by rind crack between 10 to 90, typically about 10 to 30, i.e. no or very little (insignificant) tendency to crack.

In some embodiments, the triploid hybrid watermelon plant disclosed herein, is characterized by rind thickness between 4 mm to 15 mm, at times, 5 mm to 10 mm.

In some embodiments, the triploid hybrid watermelon plant disclosed herein, is characterized by vigor of between 40 and 90.

In some embodiments, the triploid hybrid watermelon plant disclosed herein, is characterized by fruit shape in the range of 50 to 60 (namely, round or slightly oval).

In some embodiments, the triploid hybrid watermelon plant disclosed herein, is characterized by fruit weight of between about 2 to 6 kg, typically about 2.5-3.5 kg, which is referred to as mini fruit size. In some other embodiments, the fruit size is midi, having a weight between 4 to 7 kg, at times, between 4-5 kg. In some embodiments, the fruit size is mini, midi or large, at least 7 kg.

In some embodiments, the triploid hybrid watermelon plant disclosed herein, is characterized by good fruit taste as determined by a human subject tasting the fruit as being non-bitter and tasteful.

In some embodiments, the triploid hybrid watermelon plant disclosed herein, is characterized by total soluble solids (referred to herein TSS) in Brix % of at least 9%, at times at least 10%, at times, between 10 to 14%, and further at times, between 10 to 13%.

In some embodiments, the triploid hybrid watermelon plant disclosed herein, is characterized by weight of a 1,000 seeds being between 30 to 70 g, at times, 30 to 40 g, at times between 32 to 36 g.

According to the present disclosure, tolerance (or lack of sensitivity) is ranked between 1 to 9, with 1 defining a high degree of sensitivity (no tolerance) that can even result in necrosis, and 9 defining high tolerance or even resistance. Thus, it is be understood that a sensitive plant has low tolerance to WmCSV and lack of sensitivity is indicative of tolerance or resistance to WmCSV. Accordingly, in the context of the present disclosure a plant ranked between 6-7 is regarded as tolerant, a plant ranked 7-8 is regarded with high tolerance or intermediate resistance, and a plant ranked 8-9 is regarded as resistant.

When referring to “genetic background” in the context of the present invention it is to be understood to refer to a cultivated watermelon plant as disclosed herein (the diploid, tetraploid or the triploid plant) containing a genome comprising at least a qualitative trait locus (QTL) which contributes to WmCSV tolerance.

In some embodiments, the QTL is genetically linked to at least one marker locus that co-segregates with the WmCSV tolerance trait and can be identified by a DNA profiling analysis technique, such as, but not limited to, PCR analysis (Polymerase Chain Reaction), SRAP analysis (Sequence-related Amplified Polymorphism) analysis, SSR analysis (Simple Sequence Repeat), AFLP analysis (Amplified Fragment Length Polymorphism) and RAPD analysis (Random Amplified Polymorphic DNA), SNP analysis (Single Nucleotide Polymorphism).

In some embodiments, the QTL is genetically linked to at least one marker locus that co-segregates with the WmCSV tolerance trait and can be identified by a PCR oligonuclotide primer or a pair of PCR oligonucleotide primers.

In some embodiments, the triploid hybrid watermelon plant comprises a tolerance locus genetically linked to at least one marker locus which co-segregates with WmCSV tolerance trait and comprises a marker that can be identified in a PCR reaction by amplification of a DNA fragment with a pair of PCR oligonucleotide primers represented by a forward primer and a reverse primer or any other marker located on chromosome 6 and is statistically correlated and genetically linked to the WmCSV tolerance trait.

Overall WmCSV-C.c. introgression performed is displayed graphically depicting a tolerance locus genetically linked to at least one marker locus which co-segregates with WmCSV tolerance trait and comprises a marker that has been identified for the tolerance to WmCSV virus is conferred by an introgression of genomic DNA of Citrullus colocynthis on the chromosome 6 of Citrullus lanatus. Subsequent to WGS analysis and as shown hereinbelow, the introgression of Citrullus colocynthis into the Citrullus lanatus genome is substantially between nucleotides 830,000-2,400,000.

Preferably, the introgressed sequences are sequenced by way of High Throughput Sequencing (HTS), also termed Next Generation Sequencing (NGS), thereby readily facilitating sequencing the entire genome substantially at once.

Preferably, HTS includes next-generation “short-read” and third-generation “long-read” sequencing methods which applies to exome sequencing, genome sequencing, genome resequencing, transcriptome profiling (RNA-Seq), DNA-protein interactions (ChIP-sequencing), and epigenome characterization.

According to a preferred embodiment, the triploid hybrid watermelon plant comprises a tolerance locus genetically linked to at least one marker locus which co-segregates with WmCSV tolerance trait and comprises a marker that has been identified for the tolerance to WmCSV virus is conferred by an introgression of genomic DNA of Citrullus colocynthis on the chromosome 6 of Citrullus lanatus. Subsequent to WGS analysis, the introgression of Citrullus colocynthis into the Citrullus lanatus genome is substantially between nucleotides 1-830,000.

According to a preferred embodiment, the triploid hybrid watermelon plant comprises a tolerance locus genetically linked to at least one marker locus which co-segregates with WmCSV tolerance trait and comprises a marker that has been identified for the tolerance to WmCSV virus is conferred by an introgression of genomic DNA of Citrullus colocynthis on the chromosome 6 of Citrullus lanatus. Subsequent to WGS analysis, the introgression of Citrullus colocynthis into the Citrullus lanatus genome is substantially between nucleotides 830,000-880,000.

According to a preferred embodiment, the triploid hybrid watermelon plant comprises a tolerance locus genetically linked to at least one marker locus which co-segregates with WmCSV tolerance trait and comprises a marker that has been identified for the tolerance to WmCSV virus is conferred by an introgression of genomic DNA of Citrullus colocynthis on the chromosome 6 of Citrullus lanatus. Subsequent to WGS analysis, the introgression of Citrullus colocynthis into the Citrullus lanatus genome is substantially between nucleotides 880,000-1,650,000.

According to a preferred embodiment, the triploid hybrid watermelon plant comprises a tolerance locus genetically linked to at least one marker locus which co-segregates with WmCSV tolerance trait and comprises a marker that has been identified for the tolerance to WmCSV virus is conferred by an introgression of genomic DNA of Citrullus colocynthis on the chromosome 6 of Citrullus lanatus. Subsequent to WGS analysis, the introgression of Citrullus colocynthis into the Citrullus lanatus genome is substantially between nucleotides 1,600,000-2,255,000.

The introgressed sequences are preferably to be found on chromosome 6 in the C. lanatus genome and thus confer tolerance to geminiviruses when they are present on every homologous chromosomes 6. The introgressed sequences conferring the tolerance are more preferably located within the chromosomal region of chromosome 6 which is delimited between nucleotides 830,000 and 2,400,000.

The introgressed sequences are preferably to be found on chromosome 6 in the C. lanatus genome and thus confer tolerance to geminiviruses when they are present on every homologous chromosomes 6. The introgressed sequences conferring the tolerance and/or resistance are more preferably located within the chromosomal region of chromosome 6 which is delimited between nucleotides 880,000 and 1,650,000.

The introgressed sequences are preferably to be found on chromosome 6 in the C. lanatus genome and thus confer tolerance and/or resistance to geminiviruses when they are present on every homologous chromosomes 6. The introgressed sequences conferring the tolerance and/or resistance are more preferably located within the chromosomal region of chromosome 6 which is delimited between nucleotides 1,600,000 and 2,255,000.

The present inventors have identified that introgressed sequences essential for the phenotype of interest, i.e. tolerance and/or resistance are to be found in the vicinity of the chromosomal region of chromosome 6 which is delimited between nucleotides 1,600,000 and 2,255,000 or in the vicinity of the chromosomal region of chromosome 6 which is delimited between nucleotides 880,000 and 1,650,000, or in the vicinity of both regions. Preferably the introgressed sequences from Citrullus colocynthis are to be found in a locus encompassing the position of between nucleotides 880,000 and 1,650,000, or in a locus encompassing the position of between nucleotides 1,600,000 and 2,255,000, or in a locus encompassing both the position of between nucleotides 880,000 and 1,650,000 and the position of between nucleotides 1,600,000 and 2,255,000. It is to be noted that the loci of between nucleotides 880,000 and 1,650,000 and between nucleotides 1,600,000 and 2,255,000 are located within the chromosomal region defined above.

The triploid watermelon plant disclosed herein is produced by crossing a WmCSV tolerant diploid male line (tolerant to resistant), seeds or offspring thereof; with a WmCSV tolerant (tolerant to resistant) tetraploid female line, seeds or offspring thereof.

Yet the present disclosure provides any offspring (progenies) asexually reproduced from the watermelon plant or seeds disclosed herein.

The reproduction of triploid offspring can be by any technique known in the art, including, without being limited thereto, cutting, tissue culture, embryo rescue, chromosome doubling, double haploids and mutations. Namely, the offspring is characterized by a reproduction of the triploid offspring by a technique selected from the group consisting of a cutting, a tissue culture, an embryo rescue, a chromosome doubling and double haploids and mutations.

Some methods are described by Wang & You, Gursoy et al; [Cucurbitaceae, 2012].

The present disclosure also provides seedless watermelon fruit obtained from the triploid hybrid watermelon plant disclosed herein or from its seeds. The seedless watermelon fruit of the triploid hybrid watermelon plant disclosed herein or of its seeds or offspring, is WmCSV tolerant and is characterized by one or more of the following parameters:
rind width between 4 mm to 15 mm, at times between 5 mm to 10 mm;
fruit shape in the range of 50 to 60 (namely, round or slightly oval; at times, essentially round or essentially oval);
fruit rind color of crimson or crimson dark; at times the fruit rind color is characterized by dark green or gray stripes or tiger-type stripes;
red flesh, preferably, dark red flesh, i.e., identified according to the Pantone Scale color as Pantone 18-1664 TCX Fiery Red (i.e., dark red) to Pantone Red 032C (i.e., light red);
mini, midi or large size; namely, the seedless watermelon fruit disclosed herein obtained from the triploid hybrid watermelon plant has fruit weight of between about 2 to 6 kg, typically about 2.5-3.5 kg, which is referred to as mini fruit size; at times, the fruit size is midi, having a weight between 4 to 7 kg, at times, between 4 to 5 kg; at times the fruit size is large, having a weight above 7 kg;
good fruit taste as determined by a human subject tasting the fruit as being non-bitter and tasteful;
total soluble solids (referred to herein TSS) in Brix % of at least 9%, at times at least 10%, at times, between 10 to 14%, and further at times, between 10 to 13%.

In general, triploid hybrid watermelon plants are created by crossing a diploid male parent line with a tetraploid female parent line. The diploid male parent line and the tetraploid female parent line are typically planted in a field or greenhouse and the female tetraploid flower is pollinated as known in the art. The triploid seeds that are produced in the fruit of the tetraploid plants are planted in a field with diploid plants to produce seedless fruits.

Thus, in another of its aspects, the invention provides a tetraploid watermelon female plant characterized by having tolerance to WmCSV and within a score tolerance scale from 1 to 9, and by having seed yield of at least 4, at times at least 5, at times at least 6, and further at times, at least 7, at times at least 8. In the context of the present disclosure, the seed production ability is to be understood as the overall fruit seed production ability of the female lines determined according to the number of seeds inside the female fruit, in a scale between 1 to 9, where 1 defines low seed production ability and 9 defines very high seed production. Preferably, a WmCSV tolerance of at least 6 (also referred to as intermediate resistance), typically between 7-9 is achieved.

In some embodiments, the genome of the tetraploid watermelon line is characterized by genetic background of a parent having at least tolerance to WmCSV, at times tolerance to WmCSV.

In some embodiments, the tetraploid hybrid watermelon plant comprises a tolerance locus genetically linked to at least one marker locus which co-segregates with WmCSV tolerance trait and comprises a marker that can be identified in a PCR reaction by amplification of a DNA fragment with a pair of PCR oligonucleotide primers represented by a forward primer and a reverse primer or any other marker located on chromosome 6 and is statistically correlated and genetically linked to the WmCSV tolerance trait

In some embodiments, the triploid hybrid watermelon plant comprises a tolerance locus genetically linked to at least one marker locus which co-segregates with WmCSV tolerance trait and comprises a marker that has been identified for the tolerance to WmCSV virus is conferred by an introgression of genomic DNA of Citrullus colocynthis on the chromosome 6 of Citrullus lanatus. Subsequent to WGS analysis, the introgression of Citrullus colocynthis into the Citrullus lanatus genome is substantially between nucleotides 830,000-2,400,000.

According to a preferred embodiment, the triploid hybrid watermelon plant comprises a tolerance locus genetically linked to at least one marker locus which co-segregates with WmCSV tolerance trait and comprises a marker that has been identified for the tolerance to WmCSV virus is conferred by an introgression of genomic DNA of Citrullus colocynthis on the chromosome 6 of Citrullus lanatus. Subsequent to WGS analysis, the introgression of Citrullus colocynthis into the Citrullus lanatus genome is substantially between nucleotides 880,000-1,650,000.

According to a preferred embodiment, the triploid hybrid watermelon plant comprises a tolerance locus genetically linked to at least one marker locus which co-segregates with WmCSV tolerance trait and comprises a marker that has been identified for the tolerance to WmCSV virus is conferred by an introgression of genomic DNA of Citrullus colocynthis on the chromosome 6 of Citrullus lanatus. Subsequent to WGS analysis, the introgression of Citrullus colocynthis into the Citrullus lanatus genome is substantially between nucleotides 1,600,000-2,255,000.

In some embodiments, the tetraploid watermelon plant of (ii) having a tolerance to WmCSV of at least 6 in a tolerance scale of 1 to 9 and low seed yield.

The present disclosure also concerns seeds of a tetraploid female watermelon plant as well as any other offspring of the tetraploid watermelon characterized by having tolerance to WmCSV, and by having seed yield of at least 4, at times at least 5, at times at least 6, and further at times, at least 7, at times at least 8.

Thus, in the context of the present disclosure, when referring to tetraploid watermelon line having said tolerance to WmCSV, it is to be understood as encompassing also any sexually or asexually reproduced offspring of the above recited tetraploid female watermelon plant lines.

Further in accordance with this aspect, there is provided pollen of a tetraploid watermelon plant disclosed herein.

The present disclosure also provides watermelon fruit obtained from the tetraploid watermelon plant line disclosed herein or from its seeds.

To create tetraploid female watermelon line, it is known in the art to use chemicals, such as colchicines, oryzaline, ethalfluralin and dinitroanilines, that are used for doubling of existing chromosome content of a diploid inbred line that are eventually obtained [Kihara, 1951].

The diploid line used to create a tetraploid line is selected based on the desired traits for the tetraploid line.

Pollination may be insect pollination or hand pollination. In accordance with some embodiments, the pollination is hand pollination. Pollination occurs in anthesis (anthesis is the period during which a flower is fully open or sexually functional). The male flowers are collected and the pollen spread on the female flower stigma by hand. In some other embodiments, pollination is by insects (mostly bees).

Cross pollination between a tetraploid line, the female parental line, and a diploid line, the male parental line, are accomplished by either hand or insects pollination, mostly hand pollination. In hand pollination, male flowers of the diploid parent line are collected in the early morning before the visit of insects and female flower buds are uncovered where pollination with the collected fresh male flowers is performed, e.g. by hand.

The tetraploid watermelon line disclosed herein and/or fruit obtained from said tetraploid watermelon or from its seeds, may be further characterized by one or more of the following parameters. Each parameter below may constitute an independent embodiment of the present disclosure or may be combined with one or more other parameter from this list:
vigor of between 60 and 90, typically about 90;
flower sex being monoecious (M);
fruit shape in the range of 50 to 60 (namely, round or slightly oval);
small, midi or large size;
flesh color being red to deep/dark red, typically deep/dark red;
rind thickness between 4 mm to 15 mm;
fruit taste being good;
seed color being brown, dark brown or black, typically dark brown;
WmCSV tolerance of at least 6 (also referred to as intermediate resistance), typically between 7-9;
high seed production (yield) of at least 6, at times at least 7, at times, at least 8, and further at times at least 9.

As detailed above, triploid hybrid watermelon plants are created by crossing a diploid male parent line with a tetraploid female parent line, to produce seedless fruits.

In some embodiments, the diploid watermelon plant disclosed herein has tolerance to WmCSV of at least 6 in a tolerance scale between 1 to 9.

In accordance with this aspect, there is also provided diploid watermelon plant as well as any other offspring of the diploid watermelon. Thus, in the context of the present disclosure, when referring to diploid watermelon line having said tolerance to WmCSV, it is to be understood as encompassing also any sexually or asexually reproduced offspring of the above recited diploid watermelon plant lines.

Further in accordance with this aspect, there is provided pollen of a diploid watermelon plant disclosed herein.

The diploid watermelon line disclosed herein may be further characterized by one or more of the following parameters. Therefore, while being presented herein as a list, each parameter below constitutes an independent embodiment of the present disclosure and may be combined with one or more other parameter from this list:
vigor of between 60 and 90, typically about 90;
flower sex being monoecious (M);
number of fruits per plant in the range of 2 to 4;
fruit shape in the range of 50 to 60 (namely, round or slightly oval);
fruit weight of between 2 to 7 kg, typically 3 kg;
flesh color being red to deep/dark red, typically deep/dark red;
flesh firmness between 60 to 80, typically about 70;
rind thickness between 4 mm to 15 mm;
fruit taste being good;
seed color being brown, dark brown or black, typically dark brown;
weight of a 1,000 seeds being between 30 to 70 g, typically between 32-36 g;
WmCSV tolerance of at least 6 (also referred to as intermediate resistance), typically between 7-9.

In yet a further aspect, there is provided by the present disclosure a method of producing a triploid hybrid watermelon plant characterized by:
tolerance to Watermelon Chlorotic Stunt Virus (WmCSV);
being a triploid plant;
seedless fruit having a total soluble solid of at least 9%, at times between 10 to 13%; and
red fruit flesh color, preferably dark red,

the method comprising:
a. planting a tetraploid watermelon line being at least WmCSV tolerant and having high seed yield to obtain a tetraploid watermelon plant;
b. crossing said tetraploid line with a WmCSV tolerant diploid watermelon line to produce triploid seeds; and
c. planting said triploid seeds to produce triploid hybrid watermelon plant disclosed herein.

In some embodiments, the tetraploid watermelon plant of having a tolerance to WmCSV of at least 6 in a tolerance scale of 1 to 9 and having low seed yield.

After the triploid is planted, the plants are allowed to grow until harvesting at an appropriate time after flowering. In some embodiments, the method further comprises a step of harvesting triploid seedless watermelon fruit from said triploid hybrid watermelon plant of step (c). In such embodiments, the harvesting duration is about 50-70 days during the hottest of the four temperate seasons and over 90 days in the coldest of the four temperate seasons.

Pollination of the triploid plants needs a pollenizer and insects visits (mostly bees) in order to carriage the pollen grains from the pollenizer male flowers to the triploid female flowers.

In some embodiments, the method of producing the tetraploid watermelon plant disclosed herein, wherein said WmCSV tolerant tetraploid female line originating from a tetraploid female selected from the group consisting of: low seed yield tetraploid female, high seed yield tetraploid female and mixtures thereof.

### Production of Seedless Triploid Watermelons Resistant to WmCSV

The goal of the present breeding was to develop a watermelon hybrid tolerant to WmCSV that has desirable characteristics.

Origene Seeds watermelon line (a backcross between cultured watermelon line, Citrullus lanatus and wild watermelon line, Citrullus colocynthis (C.c.), a resistant wild watermelon to WmCSV) is tolerant to WmCSV. The wild watermelon diploid C.c. line is identified herein PI 418 (selection for virus tolerance in a heterozygous line) The fruits of the wild watermelon line are characterized by being small, white flesh, with hard seeds and bitter taste.

The selection method for finding the tolerance plants was based on infecting by whitefly (i.e., small insects that typically feed on plant leaves). Specifically, Bemisia tabaci whitefly specie, acquired WmCSV from infected plants, transmitted the virus to healthy watermelon seedlings which developed typical WmCSV symptoms 10-15 days thereafter.

The tolerant plants were selected and chosen and propagated for more cycles of tolerance enhancement for improving tolerance to WmCSV.

Backcross breeding program that included the most tolerant plants and cultivated plants with the desired characteristics was developed to obtain a tolerant hybrid with the desired characteristics.

The triploid watermelon hybrids were produced by cross of a tetraploid WmCSV tolerant line as the female and a diploid WmCSV tolerant line as the male.

The breeding program period for obtaining a triploid watermelon hybrid tolerant to WmCSV was 8 years during the years 2005-2012, in two seasons per year, winter and summer.

### Example 1: Development Stages of the Diploid Male (and Timeline): From F1 Till BC3F15

### Diploid Male Parent Line Development

The aim of the present breeding was to develop a watermelon hybrid tolerant to WmCSV that has desirable characteristics.

All of the breeding stages were conducted in a greenhouse in Bnei Darom Research Farm in Israel.

Citrullus colocynthis (referred to herein as “C.c.”), were used as the source tolerant to WmCSV. Ten seeds of Citrullus colocynthis were seeded in Hishtil nurseries and this line was artificially infected by whitefly in order to test the tolerance of the plants to WmCSV. Specifically, the plants were infected by transmitting the virus from Bemisia tabaci, a species of whitefly, acquired WmCSV from infected plants to healthy watermelon seedlings. WmCSV infected plants typically develop WmCSV symptoms 10-15 days after infection, recognized as yellow spots appearing on the apex of the plant and on the young leaves above the inoculation point. With time, the chlorotic spots spread to the rest of the plant. The plants of the Citrullus colocynthis showed no symptoms.

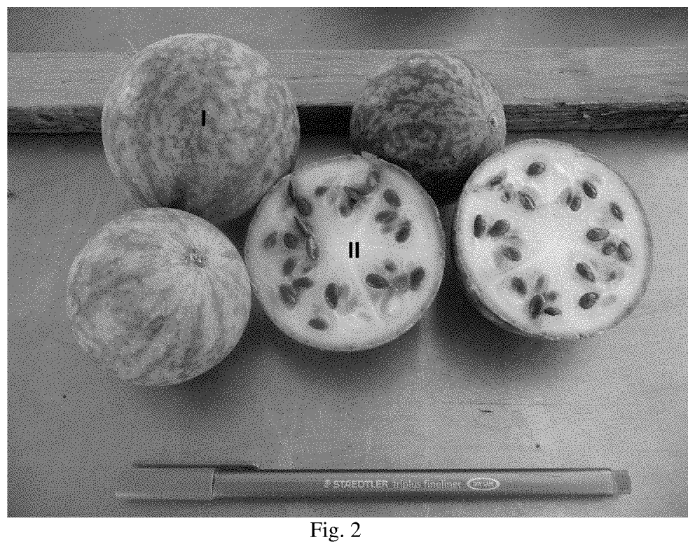

C.c. is a wild watermelon with undesirable traits such as bitter taste and white flesh, stripped rind (See (I) in FIG. 2), white flesh and brown seeds (See (II) in FIG. 2).

Therefore, in order to obtain an edible fruit with desired characteristics, an inventive backcross breeding process, using cultured lines was conducted, to inherit the desired characteristics.

### Development Stages of the Diploid Male and Timeline:

As previously presented above, in view of the undesirable traits of the C.c. line fruit, such as, bitter taste and white flesh, in order to obtain an edible fruit with desired characteristics, a backcross breeding procedure using cultured lines was conducted from 2005.

### Characterization of the Diploid Male Parent Lines:

The diploid male parent lines obtained are characterized by the following parameters which are displayed in Table 2:

### Fruit Properties

Shape of fruit: round or slightly oval (and are referred to in Table 2 as having a fruit shape value between 50-55);
Rind properties: Dark tiger color (i.e., green rind with dark green narrow stripes, 5-8 mm thickness;
Flesh properties: red to dark red;
Taste: Good;
Weight of fruit: about 3 kg;
Seeds: Dark brown color, TSW in the range of 32-36 g.

### Plant Properties

Vigor: the plants of the four generations of the same line exhibited a vigor value of 90 indicating a very strong plant.

### WmCSV Tolerance to Tolerance

In a scale from 1 to 9 (9 being the best performance of WmCSV tolerance), the male parent lines exhibited tolerance to WmCSV of 7.

In conclusion, a tiger stripped rind of 5 mm width, round shape, red flesh, dark brown medium size seeds (average seed size of 5 mm*8 mm), very strong plant and WmCSV tolerant was obtained for the male lines described herein above.

May 2005 planting—WmCSV infected and survived plants of the line “C.c.” (as a male parent) and plants of Origene Seeds proprietary line “112” (as a female parent) were planted in a greenhouse.

August 2005 F1—Seeds obtained from a cross between the C.c. line (male parent) and “112” line (female parent) by hand pollination, were harvested in August 2005 and named “419-F1”.

As previously presented above, in view of the undesirable traits of the C.c. line fruit, such as, bitter taste and white flesh, in order to obtain an edible fruit with desired characteristics, a backcross breeding procedure using cultured lines was conducted.

July 2005 BC1F1—A first backcross breeding of a single plant of “419-F1” and “112” (proprietary of Origene Seeds Ltd.) was created line “419BC1-F1”. The seeds were harvested in November 2005.

February 2006 BC1F2—Line “419BC1-F1” was self-pollinated in a greenhouse providing line “419BC1-F2”.

July 2006 BC2F1—A second backcross breeding between “112” line (proprietary of Origene Seeds Ltd.) and “419BC1-F2” plants which display WmCSV tolerance, created line “419BC2-F1”. The seeds were harvested in November 2006.

February 2007 BC3F1—Line “419BC2-F1” was self-pollinated in a greenhouse providing line “419BC2-F2”. A third backcross between flowers of “419BC2-F1” plants and second Origene Seeds proprietary line “422” (cross between a sensitive line “113” and a resistant line “420-1-1”), created line “549-BC3-F1”. The seeds were harvested in May 2007.

July 2007—February 2014 BC3F2-BC3F15—Further selections were conducted in the greenhouse in order to achieve uniformity and in order to obtain a fruit with desirable characteristics: “549-BC3-F2”-“549-BC3-F15.

(*) Only plants displaying WmCSV tolerance trait in the lab (WmCSV tolerant) were used for further selections.

### Example 2: Development Stages of the Tetraploid Female and Timeline: From F1 Till BC1F11

The diploid line used to create a tetraploid was selected based on the desired tolerance to WmCSV and on characteristics for the tetraploid line.

The tetraploid line “645” was obtained from a cross between two Origene Seeds proprietary lines, “367-a” (cultured sensitive tetraploid line) and “623-3-6-1”, whereby line “623-3-6-1” was obtained by doubling of a diploid cross of line “419-1” (line “112”, proprietary line of Origene Seeds Ltd. and C.c. line being the resistance source, collected in southern areas in Israel). The chromosome doubling of line “623-3-6-1” was carried out in February 2007.

The first cross of tetraploid line “645” (referred herein as “W08-F1-645-B1”) was harvested in November 2008 after two generations of self-pollination to line “623-3”.

(*) Only plants displaying WmCSV tolerance trait in the lab were used for further selections.

TABLE 1
Development of new triploid hybrid lines tolerant to WmCSV

| Triploid hybrid | Tetraploid female line | Diploid male line |
|---|---|---|
| line | resistant to WmCSV | resistant to WmCSV |
| 12.607-S12 | WmW11-F06-1039-BR1 | WmW11-F10-1025-B1 |
| 12.607-W13 | WmS13-F09-862-BR1 | WmS13-F13-838-B1 |
| 12.607-S14 | WmW13-F10-1146-BR1 | Wm2W13-F14-1115-B1 |
| 12.607-W14 | WmS14-F11-943-B1 | Wm2S14-F15-916-SB1 |

### Example 3: Development of Triploid Hybrids Tolerant to WmCSV

The particular selection of the female and male allowed the transfer of the tolerance to the triploid hybrid. The unique selection of the female line with tolerance to WmCSV with a male diploid line with high WmCSV tolerance generated a WmCSV tolerant triploid hybrid with unique properties.

The triploid hybrids were obtained from a cross between two sources having WmCSV tolerance, namely between WmCSV tolerant tetraploid female lines and WmCSV tolerant triploid male lines. The resulting series of WmCSV tolerant triploid hybrid 12.607 is illustrated in Table 1 and named: “12.607-S12”, “12.607-W13”, “12.607-S14” and “12.607-W14”.

### Characterization of the Triploid Hybrid:

The characteristics of the triploid hybrid with tolerance to WmCSV, “12.607-S12”, “12.607-W13”, “12.607-S14” and “12.607-W14”, are provided in Table 2.

### Fruit Properties

Shape of fruit: round or slightly oval;
Rind properties: Crimson dark color, dark stripes and 10 mm thickness;
Flesh properties: Red or dark red color;
Taste: Good;
Weight of fruit: about 2.5 kg.

### Plant Properties

Vigor: the plants exhibited a vigor value of about 40;

### WmCSV Tolerance to Tolerance

In a scale from 1 to 9 (9 being the best performance of WmCSV tolerance), the hybrid lines exhibited tolerance to WmCSV of 7.

In conclusion, a tiger stripped rind of 10 mm width, round shape, red flesh, strong and WmCSV tolerant plant was obtained for the new hybrid triploid lines described herein above.

TABLE 2
Characteristics of the triploid hybrids

| Hybrid line | 12.607-S12 | 12.607-W13 |
|---|---|---|
| Vigor of plant | 40 | 40 |
| Fruit rind color | Crimson dark | Crimson dark |
| Fruit shape | 50 (Round) | 50 (Round) |
| Fruit striping | Dark stripes | Dark stripes |
| Fruit weight [kg] | 2.5 | 2.5 |
| Flesh color | Red | Deep red |
| Fruit rind thickness [mm] | 10 | 10 |
| Fruit taste | Good | Good |
| WmCSV tolerance | 7 | 7 |

Further selections to obtain desirable characteristics of the tetraploid female line, such as: uniformity, fruit's quality and high seed production ability were made in the greenhouse as follows:

The resulting series of WmCSV tolerant triploid hybrid OCR7 is named “OCR7131”, “OCR7132”, “OCR7133”, “OCR7134”, “OCR7135”, “OCR7136”, “OCR7137”, “OCR7138”, “OCR7139”, “OCR7140”, “OCR7141”, “OCR7142”, “OCR7143”, “OCR7144” and “OCR7145”.

The triploid hybrid OCR7 series of the present invention have the desired watermelon properties, such as, but not limited to, WmCSV tolerance, dark red flesh, good taste and high vigor.

The newest and latest hybrids series is named “ORS60601-ORS60602”, “ORS60605-ORS60606”, “ORS7235-ORS7236”, “ORS7241”, “ORS70323-ORS70332”, “ORS70334-ORS70335”, “BONECI” and “ORS70330”, (ATCC xxx-ATCC xxx).

Surprisingly it was found that the triploid watermelon hybrid is not tolerant to WmCSV if only one parent is tolerant.

### BACKGROUND ART

References considered to be relevant as background to the presently disclosed subject matter are listed below:
Abudy A, Sufrin-Ringwald T, Dayan-Glick C, Guenoune-Gelbert D, Livneh O, Zaccai M, Lapidot M, 2010, Watermelon chlorotic stunt and Squash leaf curl begomoviruses—new threats to cucurbit crops in the Middle East, Israel J Pl Sci
Al Musa A, Anfoka G, Al Abdulat A, Misbeh S, Haj Ahmed F, Otri I, 2011, Watermelon chlorotic stunt virus (WmCSV): a serious disease threating watermelon production in Jordan, Virus Gene DOI 10.7007/s11262-011-0594-8.
Alhudaib K., Soliman A. M., Rezk, A. A., 2018. Current Status of Watermelon Chlorotic Stunt Virus (WmCSV) on Some Cucurbit Plants (Cucurbitaceae) in Alahsa Region of Saudi Arabia. Sci. J of King Faisal Uni. 19 (2):37-45.
Gursoy I, Solmaz I, Deliboran S, Sari, N, 2012, In vitro ovule and ovarium culture in watermelon, Cucurbitaceae 2012, 799-804.
Kheyr-Pour A, Bananej K, Dafalla A Caciagli P, Noris A, Ahoonmanesh A, Lecoq H, Gronenborn B 2000, Watermelon Chlorotic Stunts Virus from Sudan and Iran: sequence comparisons and identification of a whitefly-transmissible determinant, Phytopatol 90:629-635.
Kihara H, 1951, Triploid watermelons, Processings of American Society of Horticultural Science 58:217-230.
Levi A, Thomas C E, Trebitsh T, Salman A, King J, Karalius J, Newman M, Reddy O U K, Xu Y, Zhang X, 2006, An extend linkage map for watermelon based on SRAP, AFLP, SSR, ISSR and RAPD markers, J Amer Soc Hort Sci, 131 (3): 393-402.
Rojas M. R., 1993, Use of degenerate primers in the Polymerase Chain Reaction to detect whitefly-transmitted Geminiviruses. Pl. Disease 77(4): 340-347.
Wang X O, You H B 2012, Study on in vitro culture of mini-watermelon, Cucurbitaceae 2012, 292-298.

What is claimed is:

1. A watermelon seed characterized by:
a) tolerance to Watermelon Chlorotic Stunt Virus (WmCSV);
b) a fruit having a total soluble solid (TSS) of at least 9%; and
c) Wherein at least one introgressed sequence conferring said tolerance is chosen from at least one introgressed sequence from C. colocynthis present on chromosome 6, in the genome of a Citrullus lanatus seed-WMCSVR, deposited at NCIMB accession number 12607 (NCIMB 42400).

2. The watermelon seed of claim 1 comprising a watermelon plant and seed, namely of Citrullus lanatus, which are resistant to geminiviruses, especially to begomoviruses including WmCSV, comprising in their genome introgressed sequences from C. colocynthis conferring tolerance to said viruses, when present homozygously.

3. The watermelon seed of claim 1 comprising a tolerance locus genetically linked to at least one marker locus which co-segregates with WmCSV tolerance trait and comprising a marker that identified for the tolerance to WmCSV virus.

4. The watermelon seed of claim 1 further comprising:
a) a tolerance locus genetically linked to at least one marker locus which co-segregates with WmCSV tolerance trait and comprising a marker that identified for the tolerance to WmCSV virus;
b) wherein said tolerance trait is conferred by an introgression of genomic DNA of Citrullus colocynthis on the chromosome 6 of Citrullus lanatus; and
c) wherein a reproduction of said Citrullus lanatus characterized by a cutting off of a part of the Citrullus lanatus embryos.

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. The watermelon seed of claim 1 comprising at least one watermelon fruit of Citrullus lanatus, resistant to geminiviruses, especially to begomoviruses including WmCSV, including a genome introgressed sequences from C. colocynthis conferring tolerance to said viruses, when present homozygously.

28. The watermelon seed of claim 82, wherein said C. lanatus seeds further includes a selection of a triploid watermelon hybrid devoid of only one parent is tolerant to WmCSV.

29. The watermelon seed of claim 3 wherein said tolerance trait is conferred by an introgression of genomic DNA of Citrullus colocynthis on the chromosome 6 of Citrullus lanatus.

30. The watermelon seed of claim 4, wherein said introgression of Citrullus colocynthis into the Citrullus lanatus genome is substantially between nucleotides 880,000-1,650,000.

31. The watermelon seed of claim 4, wherein said introgression of Citrullus colocynthis into the Citrullus lanatus genome is substantially between nucleotides 1,650,000-2,255,000.

32. The watermelon seed according to claim 31, wherein said HTS includes a next-generation “short-read” and a third-generation “long-read” sequencing method, which said sequencing is selected from the group consisting of an exome sequencing, a genome sequencing, a genome resequencing, a transcriptome profiling (RNA-Seq), a DNA-protein interactions (ChIP-sequencing), and an epigenome characterization.

33. (canceled)

34. The watermelon seed of claim 31, wherein said watermelon seed is a triploid watermelon seed and wherein said triploid watermelon seed produces seedless watermelon fruit.

35. (canceled)

36. A method of producing a watermelon seed according to claim 1, comprising:
a) crossing a virus resistant watermelon with a virus sensitive watermelon variety and selecting for further breeding a seed of a hybrid plant that is among the most virus resistant and contains in its genome an introgressed sequences located on homologous chromosome 6, within the chromosomal region selected be a delimitation by nucleotides 1-2,255,000;
b) backcrossing the selected hybrid plant produced from said seed with a virus sensitive watermelon plant and selecting for further breeding a backcross hybrid plant that is among the most virus tolerant and contains in its genome said homologous chromosome 6, within the chromosomal region selected be a delimitation by nucleotides 1-2,255,000;
c) selecting a most virus tolerant plant of step (b) for further treatment, thereby readily bringing about chromosome doubling and producing a female tetraploid virus resistant watermelon plant;
d) pollinating said tetraploid watermelon plant produced with pollen from said backcross hybrid as a male parent diploid watermelon plant to produce a plurality of triploid watermelon seeds;
e) selecting triploid watermelon seed produced by step (d) so as to plant only a seed that contains in its genome said homologous chromosome 6, within the chromosomal region selected be a delimitation by nucleotides 1-2,255,000; and
f) planting said triploid watermelon seed of step (e) to produce a triploid watermelon plant characterized by resistance or intermediate resistance to viruses.

37. The method claim 36, further including, prior to step (d) repeated said backcrossing step (b).

38. The method claim 36, wherein the product of said crossing of (a) is used as the male of backcrossing of (b).

39. The method claim 36, wherein the product of said crossing of (a) is used as the female of backcrossing of (b).

40. The watermelon seed according to claim 31, wherein said introgressed sequences are sequenced by way of High Throughput Sequencing (HTS) or Next Generation Sequencing (NGS), thereby readily facilitating sequencing the entire genome substantially at once.

41. A watermelon fruit characterized by:
a) tolerance to Watermelon Chlorotic Stunt Virus (WmCSV);
b) wherein said fruit has a total soluble solid (TSS) of at least 9%; and
c) wherein at least one introgressed sequence conferring said tolerance is chosen from at least one introgressed sequence from C. colocynthis present on chromosome 6, in the genome of a Citrullus lanatus seed-WMCSVR, deposited at NCIMB accession number 12607 (NCIMB 42400).

42. A method of producing a watermelon fruit according to claim 41, comprising:
a) crossing a virus resistant watermelon with a virus sensitive watermelon variety and selecting for further breeding a fruit of a hybrid plant that is among the most virus resistant and contains in its genome an introgressed sequences located on homologous chromosome 6, within the chromosomal region selected be a delimitation by nucleotides 1-2,255,000;
b) backcrossing the selected hybrid plant produced from said seed with a virus sensitive watermelon plant and selecting for further breeding a backcross hybrid fruit that is among the most virus tolerant and contains in its genome said homologous chromosome 6, within the chromosomal region selected be a delimitation by nucleotides 1-2,255,000;
c) selecting a most virus tolerant fruit of step (b) for further treatment, thereby readily bringing about chromosome doubling and producing a female tetraploid virus resistant watermelon plant;
d) pollinating said tetraploid watermelon plant produced with pollen from said backcross hybrid as a male parent diploid watermelon plant to produce a plurality of triploid watermelon seeds;
e) selecting triploid watermelon seed produced by step (d) so as to plant only a seed that contains in its genome said homologous chromosome 6, within the chromosomal region selected be a delimitation by nucleotides 1-2,255,000; and
f) planting said triploid watermelon seed of step (e) to produce a triploid watermelon fruit characterized by resistance or intermediate resistance to viruses.
